(19) United States
(12) Patent Application Publication
(10) Pub. No.: US 2017/0303815 A1
(43) Pub. Date: Oct. 26, 2017

(54) APPARATUSES AND METHODS FOR DETERMINING LUNG WETNESS

(71) Applicant: IMPEDIMED LIMITED, Pinkenba (AU)

(72) Inventors: Alfonso L. DE LIMON, Encinitas, CA (US); Scott M. CHETHAM, Cardiff by the Sea, CA (US); Todd Aaron KIRSCHEN, La Jolla, CA (US); Brijesh Basavaraj SIRPATIL, San Marcos, CA (US); Lee Fason HARTLEY, Carlsbad, CA (US); Christopher J. FLAHERTY, Auburndale, FL (US)

(21) Appl. No.: 15/521,551

(22) PCT Filed: Oct. 30, 2015
(86) PCT No.: PCT/AU2015/050686

§ 371 Date: Apr. 24, 2017

Related U.S. Application Data
(60) Provisional application No. 62/073,790, filed on Oct. 31, 2014.

(51) Int. Cl. A61B 5/053; A61B 5/08; A61B 5/00; A61B 2562/0215

(52) U.S. Cl. CPC A61B 5/0537; A61B 5/6841; A61B 5/0809; A61B 5/4878; A61B 5/6831; A61B 5/6833; A61B 2562/0215; A61B 2560/0412; A61B 2560/045; A61B 2562/043; A61B 2562/164

(57) ABSTRACT

Described herein are method and apparatuses (devices and systems) for determining tissue wetness, and particularly lung wetness. In particular, described herein are apparatuses including patch sensors having a plurality of electrodes one a substrate that includes alignment tabs for aiding in alignment. Also described herein are patch sensors having one or more substrate modifications to enhance local flexibility of the patch. Finally, described herein are apparatuses for determining lung wetness that determine the contour of the body region onto which the patch is applied, e.g., using a diagnostic tool to measure body contour.

### CROSS REFERENCE TO RELATED APPLICATIONS

This material may related to the following patents and patent applications, herein incorporated by reference in their entirety: U.S. Provisional Patent Application No. 62/073,790, filed on Oct. 31, 2014 (titled “APPARATUSES AND METHODS FOR DETERMINING LUNG WETNESS”); U.S. patent application Ser. No. 13/715,788, filed on Dec. 14, 2012 (titled “METHODS FOR DETERMINING THE RELATIVE SPATIAL CHANGE IN SUBSURFACE RESISTIVITIES ACROSS FREQUENCIES IN TISSUE”); U.S. patent application Ser. No. 14/171,499, filed Feb. 3, 2014 (titled “DEVICES FOR DETERMINING THE RELATIVE SPATIAL CHANGE IN SUBSURFACE RESISTIVITIES ACROSS FREQUENCIES IN TISSUE”); and U.S. Pat. No. 8,068,906, issued Nov. 29, 2011 (titled “CARDIAC MONITORING SYSTEM”).

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Apparatuses, including devices and systems, as well as methods for determining lung wetness are described herein. In particular, described herein are non-invasive methods and systems for determining lung wetness using a patch sensor (patch) including an array of electrodes having a fixed predetermined configuration are configured to conform to a subject's body to electrical properties that indicate lung wetness.

### BACKGROUND

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Tissue water content is an important and informative diagnostic parameter. Dehydration decreases cognitive and physical work capabilities, while the excessive hydration (swelling, edema) is a common symptom of cardiac, hepatic or renal pathology, malnutrition and many other pathologies and diseases. Edema causes muscle aches and pains and may affect the brain, causing headaches and irritability. Edema is a major symptom for deep venous thrombosis. It may be caused by allergies or more serious disorders of the kidney, bladder, heart, and liver, as well as food intolerance, poor diet (high sugar & salt intake), pregnancy, abuse of laxatives, diuretics, drugs, the use of contraceptive pills, hormone replacement therapy, phlebitis, etc.

For example, muscle water content (MWC) is a clinically useful measure of health. Monitoring of muscle water content can serve as an important indicator of body hydration status in athletes during the training as well as in soldiers during deployment. It is generally known that body hypohydration causes severe complications, health and performance problems, and that increasing body water weight loss causes increasing problems: water weight loss of up to 1% causes thirst, 2% may cause vague discomfort and oppression, 4% may cause increased effort for physical work, 5% may cause difficulty concentrating, 6% may cause impairment in exercise temperature regulation, increases in pulse and respiratory rate; 10% may cause spastic muscles; and 15% may cause death. Soldiers commonly dehydrate 2% -5% of body weight due to high rate of water loss from environmental exposure and performing stressful physical work. Dehydration by modest amounts (2%) decreases cognitive and physical work capabilities, while larger water losses have devastating effects on performance and health. Numerous pathologic signs and symptoms due to body dehydration include digestion problems, high blood pressure, muscle cramps, etc. MWC monitoring by an objective instrument may help prevent hazard thresholds. This is important because subjective indicators like thirst can be inadequate.

Control of MWC in athletes and soldiers could help in monitoring total body hydration during long-term endurance exercise or performance in hot weather conditions. In addition, tissue wetness may be particularly helpful in assessing lung wetness, which may be an important metric for treating cardiac disorders such as congestive heart failure.

Congestive heart failure (CHF) causes difficulty breathing because oxygen exchange in the lung is impeded by pulmonary congestion. The vast majority of CHF hospital admissions are because of difficulty breathing. Further, the high rate of CHF readmission (by some estimates approximately 24% within 30 days) is due to re-accumulation or inadequate removal of pulmonary congestion resulting in difficulty breathing. Currently, there is no quantifiable method or metric to identify pulmonary congestion and better prevent difficulty breathing and hospital admission. This problem is growing. In 2010, there was an estimated of 5.8 million CHF cases in the U.S., with over 670,000 new cases each year.

A subject suffering from CHF may be diagnosed using a physical exam and various imaging techniques to image the subject's chest. Treatment typically includes the use of vasodilators (e.g., ACEI/ARB), beta blockers, and diuretic therapy (e.g., Lasix). Management of treatment often proves difficult and unsuccessful. In particular, diuretic therapy is difficult for subjects and physicians to optimally manage. For example, changes in diet may require frequent changes in the diuretic therapy. Overuse (an underuse) of diuretic therapy may negatively impact clinical outcomes.

Pulmonary congestion is typically the result of high pulmonary blood pressures that drive fluid into the extravascular “spongy” interstitial lung tissue. High pulmonary blood pressures are present in subjects with elevated intravascular filling pressures as a result of heart failure. This high pulmonary blood pressure may also lead to increased amounts of fluid entering the extravascular space. Congestion within the extravascular interstitial lung tissue may prevent gas exchange ultimately, leading to a difficulty breathing that may require hospitalization. Hospital therapies are typically directed at reducing the pulmonary blood pressure by removing intravascular fluid with diuretic therapy. Although subject symptoms may improve, significant extravascular interstitial fluid may still be present. Subjects may feel well enough for discharge, but only a small change in pulmonary blood pressures will cause fluid to quickly re-accumulate, requiring readmission. Thus, subject symptoms do not reflect adequate treatment for the extent of the disease. Therefore, there is a need to detect and monitor extravascular interstitial fluid (e.g., lung wetness) and to provide an index or measure of the level extravascular interstitial fluid both instantaneously, and over time.

There are several methods for assessing total body water, as the most prominent indicator of hydration status, including methods based on bioelectrical impedance and conductance. For example, U.S. Pat. No. 4,008,712 to Nyboer discloses method and apparatus for performing electrical measurement of body electrical impedances to determine changes in total body water in normal and deranged states of the body, U.S. Pat. No. 5,615,689 to Kotler discloses a method of predicting body cell mass using impedance analysis, U.S. Pat. No. 6,280,396 to Clark discloses an apparatus and method for measuring subject's total body water content by measuring the impedance of the body, and U.S. Pat. No. 6,459,930 to Takehara et al. discloses a dehydration condition judging apparatus by measuring bioelectric impedance. However, these methods and systems have proven unreliable and difficult to implement. The aqueous tissues of the body, due to their dissolved electrolytes, are the major conductors of an electrical current, whereas body fat and bone have relatively poor conductance properties. Significant technical problems have hampered many such electrical methods for in vivo body composition analyses; impedance spectroscopy is an attempt to refine bioimpedance measurements, which measures resistance and reactance over a wide range of frequencies. A technique based on this approach is described in U.S. Pat. No. 6,125,297 to Siconolfi which describes a method and apparatus for determining volumes of body fluids in a subject using bioelectrical response spectroscopy.

Although various systems for using electrical energy have been proposed and developed, many of these systems are complex and difficult and expensive to implement. For example, systems such as electrical impedance imaging/tomography (EII/EIT) and applied potential tomography have been described elsewhere. For example, a system such as the one described in U.S. 2007/0246046 to Teschner et al. (and others owned by the Draeger corporation) uses an electrical impedance tomography (EIT) method for reconstituting impedance distributions. In such systems, a plurality of electrodes may be arranged for this purpose on the conductive surface of the body being examined, and a control unit, usually a digital signal processor, typically ensures that a pair of (preferably) adjacent electrodes are each supplied consecutively with an electric alternating current (for example, 5 mA at 50 kHz), and the electric voltages are detected at the remaining electrodes acting as measuring electrodes and are sent to the control unit. Typically, a ring-shaped, equidistant arrangement of 16 electrodes is used, and these electrodes can be placed around the body of a subject, for example, with a belt. Alternating currents may be fed into two adjacent electrodes each, and the voltages are measured between the remaining currentless electrode pairs acting as measuring electrodes and recorded by the control unit.

Other described EIT systems, such as those illustrated in U.S. Pat. No. 7,660,617, U.S. 2010/0228143, and WO 91/019454, do not show evidence that measurements would not vary with subject habitus, e.g., body shape or geometry.

Unfortunately, electrical impedance methods have proven difficult to reliably and accurately implement for determining tissue wetness, and particularly lung wetness. Often, additional anthropometric terms (i.e., weight, age, gender, race, shoulder width, girth, waist-to-hip ratio, and body mass index) must be included in these previous prediction models to reduce the error of the estimate within acceptable boundaries. In addition, the reliability and reproducibility of the wetness estimates may vary depending on the geometry and placement of the electrodes. Thus, current methods and systems for assessing water content based on the bioimpedance of tissues may result in low accuracy, significant dependence of testing results on the anthropometrical features of the subject and on electrolyte balance.

There is therefore a need for a simple and highly accurate method and device for monitoring tissue hydration status that can be used in a broad range of field conditions.

### SUMMARY OF THE DISCLOSURE

Described herein are method and apparatuses (devices and systems) for determining tissue wetness, and particularly lung wetness. In particular, described herein are apparatuses including patch sensors having a plurality of electrodes on a substrate that includes alignment tabs for aiding in alignment. Also described herein are patch sensors having one or more substrate modifications to enhance local flexibility of the patch. Finally, described herein are apparatuses for determining lung wetness that determine the contour of the body region onto which the patch is applied, e.g., using a diagnostic tool to measure or otherwise assess body contour.

For example, described herein are systems, devices and methods that may provide an objective measure of tissue wetness. In some specific variations, the systems, devices and methods may be configured to measure pulmonary congestion (e.g., extravascular interstitial fluid) in in-subject and/or out-subject settings, including home use. For example, the systems described herein may provide non-invasive, accurate, and reproducible measures of pulmonary congestion. These systems may be referred to as lung fluid assessment monitors. Any of the systems described herein and may include executable logic to detect tissue wetness utilizing relative percent differences of apparent resistivities from the skin into the tissue derived from applying currents and measuring voltages in a specified geometric pattern of electrodes applied to the skin. The systems described herein may therefore be non-invasive, rapid, and do not use ionizing radiation.

Some variations of the systems described herein may be referred to as lung fluid assessment monitors, and may have executable logic configured to detect extravascular interstitial lung fluid utilizing determining relative spatial change in subsurface resistivities across frequencies from the skin to the lung region derived from applying currents and measuring voltages in a specified geometric pattern of electrodes applied to the skin. As mentioned, these systems may also provide an objective absolute measurement of pulmonary fluid status, such as an extravascular lung water (EVLW) index. The systems, devices and methods described herein may address many of the problems identified above, and may offer reliable and effective techniques for determining tissue wetness by determining a distribution of relative percent differences of the tissue regions beneath the electrodes to derive a value or distribution of values that are independent of the subject's body geometry. The resulting information may provide a map indicating the relative percent differences of spatial distributions of resistivities within the body across multiple frequencies. Also described herein are methods of interpreting the relative percent difference map to determine tissue wetness and, in particular, to monitor changes in tissue wetness.

For example, an array of electrodes having a predetermined configuration for detecting lung wetness may be referred to as a patch, sensor patch or patch sensor. The sensor patches described herein may hold the plurality of electrodes in a predetermined arrangement, yet may be sufficiently flexible or conformable so that they can be self-adherent to the subject's body (e.g., back) to hold each of the electrodes in the plurality of electrodes on the patch (where a patch may be greater than 1 inch (2.5 cm) wide and 6 inches (15 cm) in length in some variations) while maintaining continuous electrical contact with the patient's body. Thus, any of the patches described may include local regions that enhance flexibility of the overall patch without compromising the fixed spatial relationship between the electrodes. The local regions that enhance flexibility may be referred as a substrate modification (or flexibility-enhancing substrate modifications). A substrate modification may be a cut-out region, a cut (e.g., slit), or generally a local region in the substrate of the patch that has a greater flexibility than the rest of the patch. In general, the substrate modifications enhance conformance of the patch sensor (electrodes) against the three-dimensional contour of a patient's body, and particularly the patient's back. The local regions of the substrate that include substrate modifications enhancing flexibility of the overall patch may reduce the lifting force resulting from the relatively rigid electrodes and other patch substrate regions when the patch sensor is applied against the subject's body, e.g., preventing the patch from lifting away from the skin when force is applied by the electrodes contacting the skin.

Any of the patch sensors (patches) including electrodes described herein may also include one or more alignment tabs for assisting a user in applying the patch on a subject in a predetermined location. For example, a patch may include one or more alignment tabs and/or alignment or positioning markings/features that may be used to aid in positioning a patch on a subject, including in particular on a subject's back.

Also described herein are diagnostic tools that may be used to determine the contour of the subject's back. These tools may be integrated into a patch or used with a patch (or independent of the patch). For example, a diagnostic tool may be configured to measure contours of the subject's body, including contours of the subject's back. Measurements taken with the diagnostic tools may be used with the system to help determine lung wetness, and/or to help align and/or position a sensor patch properly on the subject's body. For example, measurement data can be used by any of the systems described herein to determine tissue wetness.

In general, systems for measuring electrical properties (e.g., relative changes in resistivities) are described. For example, a system may include an apparatus for applying and recording electrical signals. Exemplary embodiments of these systems, including patch sensors, are provided herein including dimensions, signal parameters, etc. Also described herein are modifications or variations of the apparatus. For example, an apparatus may include a strap cradle that attaches a portion of the device (e.g., an acquisition module) to a subject, and/or a garment worn by the subject, such as a strap. The strap cradle may limit or restrict access ports when the acquisition module is worn by a patient, which may prevent incorrect use/operation of the device, and/or undesired communication.

Also described herein are other variations of patch sensors, including patches that have a visible protective layer. The protective layer may be used (e.g., in manufacturing) to protect exposed electrode surfaces.

In one broad form the present invention seeks to provide a non-invasive lung wetness patch sensor, the patch sensor comprising a substrate; a plurality of electrodes on the substrate, wherein the substrate maintains a predetermined spacing between the electrodes; and at least one substrate modification to enhance local flexibility of the substrate so that the patch sensor may conform to a contour of a subject's body, wherein the plurality of electrodes are configured to form a plurality of pairs of current-injecting electrodes and a plurality of pairs of voltage detection electrodes.

Typically the substrate modifications to enhance local flexibility of the substrate comprise at least one of cut-out regions through the substrate, slits cut through the substrate and regions of material within the substrate having a greater flexibility than the substrate.

Typically the substrate is flexible and relatively inelastic, so that the spacing between each of the electrodes remains relatively fixed as the sensor is manipulated.

The patch sensor can further comprise an adhesive hydrogel.

Typically the substrate is less than about 5 mils (0.127 mm) thick.

Typically the substrate comprises at least one of a polyester material and a polyester material and an anti-bacterial titanium oxide material.

Typically the width of the substrate is between about 0.5 inches (1.3 cm) and about 2.5 inches (6.4 cm).

Typically the plurality of electrodes comprise more than 6 elongate electrodes each having a length of between about 1.5 (3.8 cm) and about 2.5 inches (6.4 cm) and a width of between about 0.1 inches (0.3 cm) and about 0.5 inches (1.3 cm), wherein the electrodes are arranged with their lengths perpendicular to a proximal to distal axis on a subject-contacting surface of the substrate so that the electrodes extend in a line parallel to the proximal to distal axis of the substrate to form an active region that extends between about 6 inches (15 cm) and about 14 inches (36 cm) along the proximal to distal axis.

Typically the plurality of electrodes comprise more than at least one of 10 electrodes and more than 25 electrodes.

Typically the electrodes have a rectangular shape on the substrate.

Typically the electrodes comprise silver/silver chloride electrodes.

Typically the electrodes are separated by a fixed distance of between about 0.2 inches (0.5 cm) and about 0.5 inches (1.3 cm) on center down a proximal to distal length of the substrate.

In one broad form the present invention seeks to provide a non-invasive lung wetness patch sensor, the patch sensor comprising a substrate; a plurality of electrodes on the substrate, wherein the substrate maintains a predetermined spacing between the electrodes; and a plurality of alignment tabs extending from a lateral side of the substrate wherein the alignment tabs are between about 0.2 inches (0.5 cm) and about 2 inches (5 cm) long and greater than about 0.1 inches (0.3 cm) wide, wherein the plurality of electrodes are configured to form a plurality of pairs of current-injecting electrodes and a plurality of pairs of voltage detection electrodes.

Typically the alignment tabs are between about 0.1 inches (0.3 cm) and about 3 inches (7.6 cm) wide.

Typically the alignment tabs comprise an upper alignment tab and a lower alignment tab.

Typically the substrate is flexible and relatively inelastic, so that the spacing between each of the electrodes remains relatively fixed as the sensor is manipulated.

Typically the patch sensor further comprises an adhesive hydrogel.

Typically the substrate is less than about 5 mils (0.127 mm) thick.

Typically the substrate comprises at least one of a polyester material and a polyester material and an anti-bacterial titanium oxide material.

Typically the width of the substrate is between about 0.5 inches (1.3 cm) and about 2.5 inches (6.4 cm).

Typically the plurality of electrodes comprise more than 6 elongate electrodes each having a length of between about 1.5 (3.8 cm) and about 2.5 inches (6.4 cm) and a width of between about 0.1 inches (0.3 cm) and about 0.5 inches (1.3 cm), wherein the electrodes are arranged with their lengths perpendicular to a proximal to distal axis on a subject-contacting surface of the substrate so that the electrodes extend in a line parallel to the proximal to distal axis of the substrate to form an active region that extends between about 6 inches (15 cm) and about 14 inches (36 cm) along the proximal to distal axis.

Typically the plurality of electrodes comprise more than at least one of 10 electrodes and more than 25 electrodes.

Typically the electrodes have a rectangular shape on the substrate.

Typically the electrodes comprise silver/silver chloride electrodes.

Typically the electrodes are separated by a fixed distance of between about 0.2 inches (0.5 cm) and about 0.5 inches (1.3 cm) on center down a proximal to distal length of the substrate.

In one broad form the present invention seeks to provide a diagnostic tool device for measuring the surface contour of a region of a patient's body, the diagnostic tool comprising a body extending in an arch from a first contact region to a second contact region, wherein a straight line extending between the first and second contact regions forms a neutral line; and a plurality of distance measuring elements coupled to the body and configured to measure the distance from a surface beneath the arch of the body and the neutral line.

The device can further comprise a flexible member extending between the first contact region and second contact region.

The device can further comprise a handle opposite the arch.

The device can further comprise a first alignment mark on the first contact region and a second alignment mark on the second contact region.

Typically the distance measuring elements comprise sliders configured to be pushed by the surface beneath the arch of the body.

Typically the distance measuring elements comprise sliders coupled to a flexible member extending between the first contact region and second contact region.

The device can further comprise a plurality of guides on the body configured to provide an estimate of distance based on the deflection of the distance measuring elements.

The device can further comprise an electronic reader configured to read measurements from the distance measuring elements.

Typically the distance measuring elements comprise non-contact, optical distance measuring elements.

In one broad form the present invention seeks to provide a method of determining tissue wetness, the method comprising attaching a patch sensor comprising a plurality of drive electrodes and sensing electrodes to a skin surface of a subject's body; measuring a curvature of the skin surface of the subject's body; applying drive currents at a plurality of different frequencies to the drive electrodes and measuring voltages at a plurality of different sensing electrodes; determining an estimate of electrical properties for a plurality of regions beneath the patch sensor using the applied drive currents and measured voltages; and determining an estimate of tissue wetness from a frequency response of the determined electrical properties.

It will be appreciated that the broad forms of the invention and their respective features can be used in conjunction and/or independently, and reference to separate broad forms in not intended to be limiting.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows one variation of an apparatus for determining tissue wetness.

FIG. 2 illustrates one variation of a patch sensor (“patch”) including an array of driving and sensing electrodes that may be used to determine tissue wetness.

FIG. 3 is an enlarged view of another variation of a patch sensor including a plurality of substrate modification regions configured as cut-out regions to enhance flexibility.

FIG. 4 is an enlarged view of another variation of a patch sensor including a plurality of substrate modification regions configured as flexible portions having a greater flexibility than the substrate.

FIG. 5 is an exploded, cross-sectional view through one variation of a patch sensor.

FIG. 6 illustrates another variation of a patch sensor for determining tissue wetness.

FIGS. 7A and 7B schematically illustrate how an apparatus such as the apparatus for determining tissue wetness shown in FIG. 1 may be worn by a subject.

FIG. 7C illustrates one variation of a cover (cradle) for an acquisition module.

FIGS. 8A and 8B illustrate one example of a tool for determining body contour that may be used with a patch sensor.

FIG. 9 illustrates another example of a tool for determining body contour that may be used with a patch sensor.

FIG. 10 illustrates another example of a tool for determining body contour using non-contact sensors.

FIGS. 11A, 11B and 11C illustrate another example of a tool for determining body contour that may be used as part of an apparatus for determining tissue wetness.

FIG. 12A illustrates one variation of a sensor assembly that may be used as part of a tool (and in particular a two-dimensional array tool) for determining body contour, such as the tool shown in FIGS. 12B and 12C.

FIGS. 12B and 12C show a front and side view, respectively of a tool for determining body contour over a two-dimensional region of a body.

FIG. 13 illustrates a method of determining tissue wetness, including a method of attaching a patch sensor and a method of collecting data and determining an estimate of tissue wetness.

### DETAILED DESCRIPTION

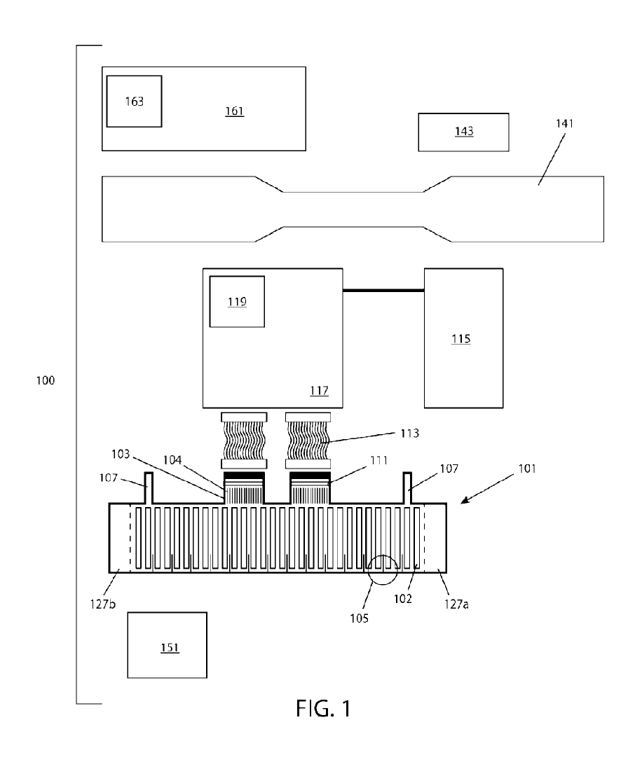

FIG. 1 illustrates one variation of an apparatus that is configured to determine lung wetness. The apparatus in this example, may measure electrical properties of biological tissue, such as conductivity or related and/or derived electrical properties, at multiple different frequencies. The apparatus may then compare how these properties vary with frequency (e.g., frequency response) to determine “wetness”, for example, by determining how similar the change in electrical response with respect to frequency is compared to that of water. For example, the more similar the frequency response of a region of tissue to the frequency response of water (e.g., saline), the more likely that the region of tissue is “wet”. Thus, this system may examine electrical properties of tissue (such as conductivity or other, related or derived electrical properties) to assess tissue (e.g. lung) wetness.

This information can then be used to derive an indicator, indicative of the wetness. This could be in the form of an absolute wetness, or relative wetness, for example compared to a baseline or other reference wetness. The indicator could additionally or alternatively, be indicative of a medical condition associated with the wetness, such as a likelihood of the subject having a condition, or a degree of a condition.

In FIG. 1, the apparatus, which is shown configured as a system 100 including multiple, interacting and/or interconnecting parts, includes a patch sensor 101 (which may also be referred to as a patch or sensor patch, each having multiple individual electrodes, or an electrode array) that connects (via connecting cables 113) to an acquisition module 117 (AM), a power supply 115 (PS), and a data analysis unit 161 (DAU). Any of the systems described herein may also include connecting cables 113 connecting the patch sensor 101 to the acquisition module 117, a patient strap 141 that can be used to hold components of the system to the patient). The system may also include a diagnostic tool 151.

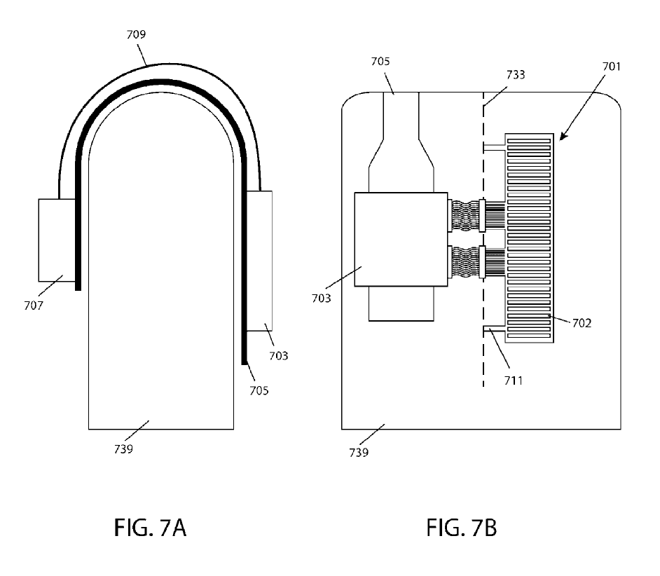

In general, many features of the patch 101 are similar to those described in U.S. Patent Application Publication No. 2013/0165761 (application Ser. No. 13/715,788) and U.S. patent application Ser. No. 14/171,499, each herein incorporated by reference in its entirety. For example, the patch 101 may include a plurality of elongate current injecting electrode pairs (simulation electrodes) and a plurality of elongate voltage sensing electrode pairs (sensing electrode pairs) which may be used sequentially or simultaneously to apply current/voltage and to sense a resulting current/voltage from which electrical properties (e.g., regional electrical properties) for one or more volumes of tissue beneath the patch may be determined. A patch 101 such as the one shown as an example in FIG. 1 may include multiple electrodes positioned on a substrate. In this example the electrodes are a linear array of 1×31 electrodes that extend over an approximately 11 inch (28 cm) length of substrate. The electrodes 102 can be spaced apart from each other with a pitch of at least 0.100 inch (0.3 cm), such as a pitch of approximately 0.360 inch (0.9 cm). Alternatively, in some variations, the patch may include a two dimensional grid of sensing electrodes with four (or more) “corner” stimulation electrodes, as shown and described below in FIG. 6.

The current injecting pairs and voltage recording pairs in the example shown in FIG. 1 can be similar and/or dissimilar sets of electrodes and/or electrode types. In some variations, the current injecting electrodes can be used as voltage sensing electrodes, and vice-versa. In some variations, the current injecting electrodes may have a different shape and/or size than the voltage sensing electrodes. For example, in some variations, the voltage sensing electrodes can have a smaller skin-contacting surface area than the current injecting electrodes (e.g. see FIG. 6). The electrodes are generally electrically conductive, and may be formed, for example of an electrically conducive metal, polymer, or the like, directly onto a substrate.

In general, the substrate may be a flexible material that supports the electrodes, as well as adhesive, traces, connector(s), and other elements (including circuitry) on the patch. For example, the substrate may include a flexible material supporting electrodes, traces, connectors, etc. In some variations, the substrate is a polyester or other non-conductive, flexible material. The substrate may have any appropriate dimensions. For example, the substrate may be approximately 0.003 inch (0.01 cm) thick, and may be relatively long and wide (e.g., between about 0.8 inches (2 cm) and about 5 inches (13 cm) wide, between about 0.8 inches (2 cm) and about 3 inches (8 cm) wide, between about 4 inches (10 cm) and about 16 inches (40 cm) long, between about 4 inches (10 cm) and about 14 inches (35 cm) long, between about 5 inches (13 cm) and about 13 inches (33 cm) long, etc., greater than 0.8 inches (2 cm) wide, greater than 4 inches (10 cm) long, etc.).

The patch can be relatively large (e.g., greater than 4 inches long by 1 inch wide), and can allow each (or at least a majority) of the individual electrode contacts (e.g., voltage sensing pairs, and current injecting pairs) to make good electrical contact with the body (e.g., back) of a patient in order to take accurate, reliable and reproducible readings. However, it is also important that the spacing between individual electrodes in the array have a relatively fixed predetermined relationship relative to each other (e.g., the distance between the electrodes and between the sensing and driving electrode pairs). Although a rigid substrate would best preserve the predetermined spacing relationship between the electrodes, e.g., preventing buckling, bending, or the like, the more rigid the substrates are less likely to conform to the outer surface of the patient's body in a region where readings are to be taken. Thus, there is a tradeoff between how rigid (e.g., stiff) to make the substrate and how flexible (bendable) to make the substrate.

Accordingly, in one example, the patch includes a substrate and a plurality of electrodes on the substrate which are configured to form a plurality of pairs of current-injecting electrodes and a plurality of pairs of voltage detection electrodes, with the substrate maintaining a predetermined spacing between the electrodes. Additionally the patch includes at least one substrate modification to enhance local flexibility of the substrate so that the patch sensor may conform to a contour of a subject's body.

In this regard, this arrangement allows the patch to conform to the subject's body, thereby ensuring good electrical contact with the body, whilst substantially maintaining a physical spacing between the electrodes, which in turn allows for improved measurement accuracy.

In FIG. 1, the substrate of the patch includes a plurality of modified regions of the substrate that enhance the local flexibility of the substrate in these regions. For example, in FIG. 1, the patch 101 includes a plurality of flexible portions 105 that enhanced conformation of substrate/electrodes to a patient's back.

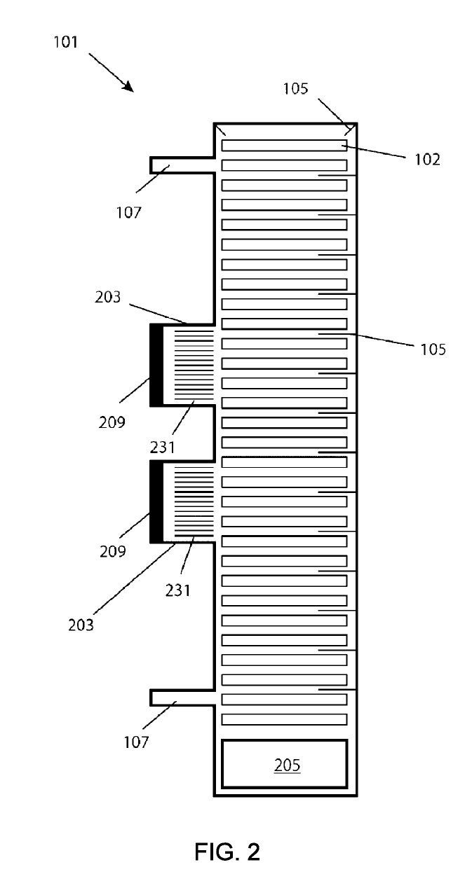

The flexible portions are shown as slits cut or formed into the substrate. In FIG. 1, the slits cut vertically from an outer elongate edge of the substrate between every other electrode 102. In FIG. 1, the slits are formed only on one side of the patch 101, for example, the side that is configured to be positioned opposite of the side of the patch that is positioned facing the spine (i.e. the side of patch 101 facing the bottom of the page as shown). FIG. 2, below, describes this in greater detail. However, it will be appreciated that alternative configurations could be used. For example, the slits could be provided on the side of the patch facing the spine, or could be provided on each side of the patch 101, depending on the preferred implementation. Additionally, the substrate modifications could be of alternative forms, such as openings, regions of different tensile elasticity or stiffness, regions of different materials, thickness or the like.

The system, and particularly the patch 101, shown in FIG. 1, can also include connecting tab portions 103. The connecting tabs 103 may be relatively stiff, such as to allow them to easily mate with connecting cables 113 or directly to the acquisition module 117 (or some other component, such as a wireless transmitter/receiver).

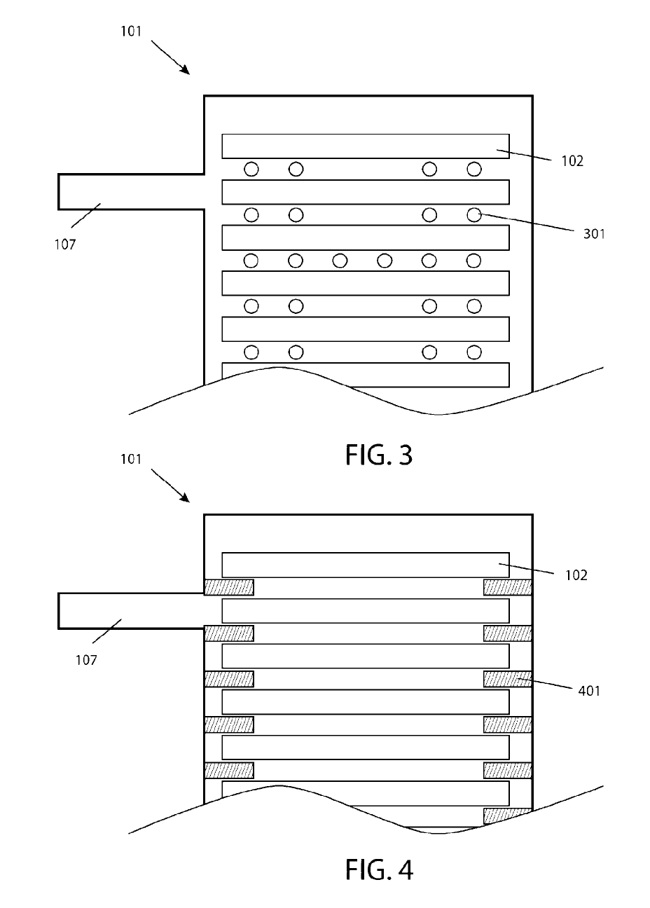

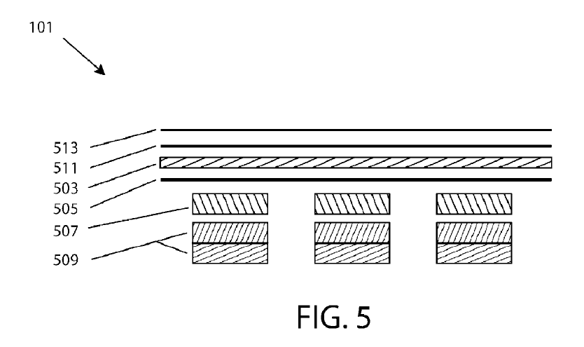

As mentioned, in FIG. 1 the flexible portions (substrate modification regions) are shown configured as slits although they may be configured generally to be regions of the substrate having an increased flexibility compared to an adjacent region. For example, in some variations the flexible portions/regions (or substrate modification regions) are cut-out regions in which shapes (e.g., circles, ovals, triangles, squares, diamonds, stars, etc.) are removed from the substrate and either allowed to be left open (see, e.g., FIG. 3), and can be filled or covered with an additional material having a greater flexibility than the rest of the substrate. In some variations the substrate may include stretchable regions, as shown and described below for FIG. 4.

In general, the individual electrodes 102 on the patch 101 may each have a surface area that is sized (e.g., is sufficiently large) to sufficiently reduce impedance encountered at electrode/patient interface. For example, electrodes 102 configured to inject current (stimulating electrodes) can comprise a skin-contacting surface large enough to avoid damage to skin and/or require high voltage drive signal. Alternatively or additionally, electrodes 102 configured for voltage or other signal sensing (sensing electrodes) can comprise a skin-contacting surface large enough to accurately record the desired signal, for example, as described briefly above, in some variations the sensor includes electrodes that are approximately 2 inches (5 cm) long, although they may be 1.5 inches (3.8 cm) long or smaller, and may be one or more order of magnitude narrower (e.g., less than about 0.2 inches (0.5 cm) wide, such as approximately about 0.160 inches (0.4 cm) wide). As mentioned, in general, the individual electrodes may be any appropriate conductive material, and may have a contact impedance of between about 10-10 kOhms, such as between 10-1000 Ohms As mentioned above, in some variations, the stimulation electrodes and the sensing electrodes may have different surface areas. For example, the stimulation electrode surface area maybe greater than the sensing electrode surface area. For example the ratio of stimulation electrode surface area to sensing electrode surface area may be greater than 5:1, 10:1, 50:1; 100:1; 1000:1, etc. The contacting surface of the electrodes (e.g., the portion of the electrode that contacts the subject's skin) could have any appropriate shape, including a shape such as rectangular (e.g. square), elliptical (e.g. circular), polygonal, etc.

In general, any of these sensors (e.g., electrodes 102) could be configured as self-adhesive electrodes and may also include one or more agents to enhance electrical contact with the subject's skin. For example, the electrodes 102 may be hydrogel electrodes. In some variations the electrodes 102 include AG603 sensing gel with a thickness of about 0.025″ (0.064 cm). In some variations, the volume resistivity of each electrode 102 is about 1000 ohm-cm maximum.

Any of the patch sensors 101 (patches) described herein may be adapted for connecting to a particular region of a patient's body, and in particular, a patient's back. Any of these patches may include one or more alignment elements, such as alignment tabs, to help align and couple the patch with a predetermined region of the subject's body.

Accordingly, in one example a non-invasive lung wetness patch sensor is provided that includes a substrate and a plurality of electrodes on the substrate configured to form a plurality of pairs of current-injecting electrodes and a plurality of pairs of voltage detection electrodes, with the substrate maintaining a predetermined spacing between the electrodes. A plurality of alignment tabs are provided extending from a lateral side of the substrate wherein the alignment tabs are between about 0.2 inches (0.5 cm) and about 2 inches (5 cm) long and greater than about 0.1 inches (0.3 cm) wide.

The use of alignment tabs allows the patch to be aligned relative to features of the subject's anatomy, such as the subject's spine. This can be used to assist in ensuring accurate and/or consistent placement of the patch on the subject. For example, this ensures the patch is positioned over the lung whose wetness is being measured, whilst ensuring that measurements are taken at the same location in the event that longitudinal monitoring is being performed.

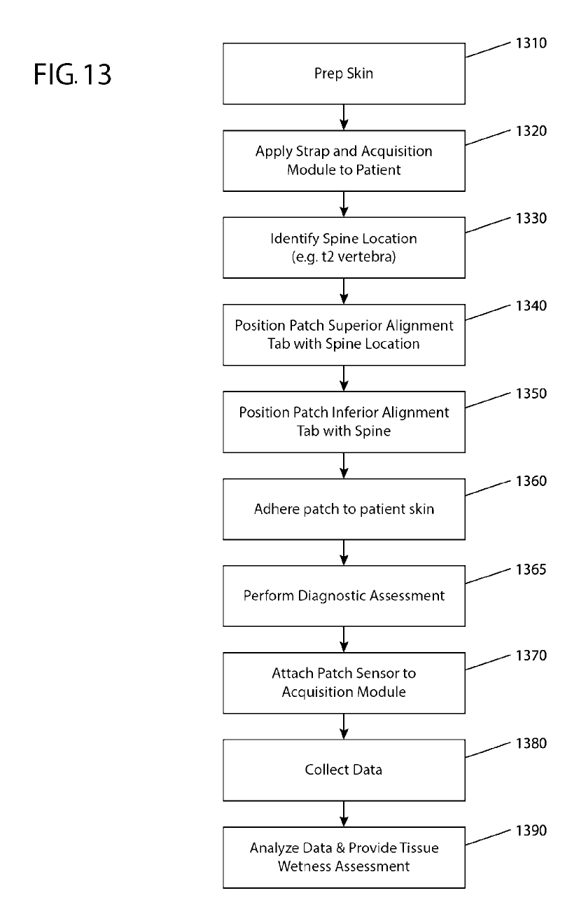

In FIG. 1 and later figures, the patch 101 includes two alignment tabs 107 that may be used to position the array of electrodes 102 relative to patient anatomy. For example, when the system 100 is adapted to measure lung wetness, the patch 101 may be positioned in a location offset from the midline of the back (the spine), at a particular height relative to the shoulders. For example, the patch 101 may include superior and inferior alignment tabs that may help a user applying the patch 101 to the subject's back to align the electrodes 102 relative to the axis of the spine (e.g., lateral to medial positioning and/or superior to inferior positioning). For example, the patch 101 may be positioned using the alignment tabs 107 to place the left edge of electrode or geometric center of electrode relative to spine so that the medial (left) edge of electrodes is approximately 1.5″ from center of spine. See, e.g., FIG. 13 for method of placement. In FIG. 1, the alignment tabs 107 are approximately 1.5 inches (4 cm) long by 0.25 inches (0.6 cm) wide, and may include one or more alignment lines, arrows or other markers on the alignment tabs 107. Patch 101 can include one or more portions that are void of electrodes, adhesive and/or other additional material, such as superior grip portion 127a and inferior grip portion 127b shown in FIG. 1. Grip portions 127a and 127b can be grasped by a caregiver or other user during placement of patch 101 on the patient's back.

As mentioned above, the patch 101 may also include one or more connecting tabs. For example, a patch 101 may include connecting tabs 103 that include traces and a connector for connection to the acquisition module 117. The connecting tabs 103 may include a flex portion/region 104 that allows the connection to move slightly (e.g. allows the acquisition module to move relative to patch 101) without disturbing the patch 101 (e.g., moving it off of the subject's body). In addition, the connecting tabs 103 may include a stiffener 111 that assists in connection with the connecting cable(s) 113. The connecting tabs 103 may include insulated traces connecting to each electrode 102 in the patch 101. In FIG. 1, the connecting tabs 103 are each about 1.6 inches (4 cm) long by about 1.6 inches (4 cm) wide. In some variations, the patch 101 and attachment components are configured for placement of a patch 101 on the right side, or on the left side, and/or may be used on either the right side or the left side of a subject's back. For example, the patch may have at distinct “top” and “bottom” or the patch 101 may be used with either end acting as the top or bottom.

Although the patch 101 show in FIG. 1 and other examples is a unitary substrate with multiple individual electrodes, in some variations the patch may comprise multiple discrete substrates (or multiple discrete patches). These patches may be connected to each other or individually connected to an acquisition module.

As shown in FIG. 1, an acquisition module 117 may connect directly or indirectly (including wirelessly) to a patch 101, and generally coordinates the application of energy (e.g., current) at different frequencies, either concurrently or sequentially, from the drive electrodes in the patch, and also coordinates the sensing of energy from the skin (e.g., sensing voltage). The energy can be supplied in one or more modes, such as a constant-current mode. In some embodiments, the supplied energy is provided while maintaining a drive voltage less than 15V, such as less than 12V, less than 10V or less than 8V. In some embodiments, the energy is supplied while maintaining the injected current at a level between a lower threshold and a higher threshold, with or without maintaining the driving voltage as described above. In general, the acquisition module 117 may include a controller, configured as an electrode drive unit (e.g., electrode drive circuitry). The electrode drive circuitry may drive multiple, different pairs of electrodes with at least two frequencies. For example, the electrode drive circuitry/unit may drive at least 2 pairs of electrodes, at least 3-16 pairs of electrodes, etc. with at least 2 drive frequencies (e.g., such as at least two or more of approximately 8 Hz, 12 kHz, 20 kHz, 50 kHz, 100 kHz and 200 kHz). The drive frequencies may be, for example, divisive submultiples of a system clock. The clock may form part of the controller forming the acquisition module. For example the drive frequencies may be divisive submultiples of a clock frequency of approximately 39 MHz. In some variations, as described in U.S. 2013/0165761, incorporated by reference above, the system (e.g., the acquisition module) operates at a lower and an upper drive frequency. For example, a lower frequency of approximately 8 kHz , 12 kHz, 20 kHz, or 50 kHz, and a higher frequency of approximately 20 kHz, 50 kHz, 100 kHz, 200 kHz, etc. As mentioned above, the energy applied can be constant current drive, constant voltage drive, or other signal that drives current from a first electrode to a second electrode through the patient. For example, an acquisition module may be configured to include a constant current source driving at between 1 mA and 10 mA, such as a current of approximately 1 mA. The apparatus may be “voltage limited”, also as described above, to avoid harm to the patient (and may include safety features to prevent overdriving. The current source may be powered by a +/−12V power supply.

In general, the applied current may be a constant current source. In some variations, the drive signal may be multiple sinusoids delivered sequentially and/or simultaneously by the patch. For example, the acquisition module 117 may be configured to deliver 2-5 simultaneously delivered different frequency sinusoids. In some variations, the apparatus may be adapted to include a common ground, e.g. a large electrode placed on patient. This may allow “monopolar” stimulation and/or “monopolar” sensing from a single electrode 102 in the patch 101. In FIG. 1, as discussed above, the patch 101 and acquisition module 117 are adapted to operate in a bipolar configuration.

An acquisition module 117 may also include a user interface 119, such as one or more of a display (including a display, touchscreen, etc.), light such as an LED, audible transducer, tactile transducer, and combinations thereof. The acquisition module may also include a control (e.g., knob, button, dial, etc.). For example, the user interface 119 may be a graphical user interface (GUI). The user interface for the acquisition module 117 may display information about the status of the acquisition module 117 or other component of system 100, and may include one or more controls for controlling activity of the acquisition module 117 or other component of system 100 (e.g., start/stop, pause/resume, inputs for user information such as height, weight, age, gender, etc.).

In general, the acquisition module 117 includes an electrode recording module (e.g., electrode recording circuitry) that allows the acquisition module 117 to record energy from the subject's skin in response to the applied energy. For example, the acquisition module 117 may record voltages from one or more pairs of the electrodes 102, including at least 1 pair, 3 pairs, 5 pairs, 10 pairs, etc. of electrodes 102.

In addition to receiving the voltage information from the patch 101, the acquisition module 117 may also correlate the received voltage with the applied energy (e.g., current), including which drive electrodes (of electrodes 102) were driven and which sensing electrodes (of electrodes 102) were used to record. The acquisition module 117 may store, transmit, process (e.g., filter, amplify, etc.) this information, and/or it may pass it directly on to a data analysis unit 161, which may be connected to the acquisition module 117 (including within the same housing) or it may be remote from the acquisition module 117.

In addition, as mentioned above, the acquisition module 117 may include an interface (e.g., interface 119) that receives subject-specific information about and/or from the subject. For example, the acquisition module 117 may include one or more inputs (e.g., buttons such as: keyboard; mouse; touchscreen; and combinations of these), and/or may receive inputs from additional measuring tools such as the diagnostic tool 151, as shown in FIG. 1. In some variations, acquisition module 117 and/or another component of system 100 can receive and/or record information such as clinician or other operator ID, Patient ID or other patient information, time, date, location, etc.

In FIG. 1 the acquisition module 117 is coupled to the patch 101 through connecting cables and may be separate from the patch 101. In some variations, the acquisition module 117 and the patch 101 are connected to each other directly. For example, at least a portion of the acquisition module 117 may be positioned on the patch; this may allow a reduction in the number of connecting wires between the acquisition module and the patch. Thus, the patch may include on-board electronics.

As mentioned and described in greater detail below, the acquisition module 117 may be integrated partially or entirely with the data analysis unit 161.

In some variations, the acquisition module 117 may include an interface or connector to one or more additional modules/devices. For example, an acquisition module 117 may include a USB Port or other data acquisition port for attachment to an external device. As mentioned, in some variations, system 100 (including the acquisition module 117) may include a wireless communication module, for wireless data transfer.

In one example, the acquisition module includes an electronic processing device, such as a microprocessor, microchip processor, logic gate configuration, firmware optionally associated with implementing logic such as an FPGA (Field Programmable Gate Array), or any other electronic device, system or arrangement, that operates to control the current source and voltage sensor. This arrangement typically includes digital to analogue converters (DACs) for coupling the processing device to amplifier for generating the required drive currents, and voltage buffer circuits coupled via analogue to digital converters (ADCs) to the electronic processing device, for returning a voltage signal.

As shown in FIG. 1, in general the apparatuses described herein include a power supply 115. The power supply 115 may be a battery or a line in (wall power) supply, or a combination of these. Power supply 115 may include capacitive power supplies, or self-generating (e.g., solar) power supplies. Power supply 115 may include a rechargeable battery or other power supply (e.g. capacitor). The power supply 115 may be integrated into the acquisition module 117 and/or the data analysis unit 161 and/or patch 101, and may include a power conditioner to condition the power for use in applying energy to the patient, including safety features, such as safety features that limit one or more of current delivered and/or voltage applied.

In general, the apparatuses described herein include a data analysis unit 161 that may receive and/or analyze the sensed electrical energy (e.g., voltage) evoked by the applied energy (e.g., current). The data analysis unit 161 typically receives information (data) from the acquisition module 117. For example, the data analysis unit 161 may upload or otherwise access information from the acquisition module 117. For example, recorded voltage data, applied drive signal data, error data and/or timing data may be received by the data analysis unit 161 from the acquisition module 117. Additionally and/or alternatively, the acquisition module could perform at least some processing of the information, for example to calculate impedance values, such as magnitudes and/or phase angle values, with the impedance values being provided to the data analysis unit.

A data analysis unit 161 may include hardware, software, firmware, or the like that is configured to operate on the received data to estimate tissue wetness, e.g., lung wetness. For example, the data analysis unit 161 may be adapted to operate on the received data and perform a tissue wetness assessment based on voltages measured from pairs of electrodes (e.g. two or more of electrodes 102) in response to multiple-frequency drive of other pairs of electrodes (e.g. two or more of electrodes 102). U.S. 2013/0165761, previously incorporated by reference, describes and illustrates one variation of a method of determining/estimating tissue wetness based on multiple frequency information. In essence, the system may determine regional electrical characteristics (such as conductivity/resistivity) for sub-regions of tissue beneath the patch at different frequencies to determine a frequency response for different regions beneath the patch. This frequency response may be compared to the frequency response for water (e.g., saline or other liquids that include water), and this comparison may be used to estimate tissue wetness. In some variations, the comparison of the frequency response may be made independently of body geometry. For example, the relative change in resistivities, which may look at the percent change in resistivities, dividing resistivity (e.g. a measured resistivity at a first location at a first frequency) by resistivity (e.g. a measured Resistivity at the first location at a second, different frequency) resulting in a “unit less” measure that may be independent of body geometry. Alternatively, in some variations the estimate of the frequency response may use body geometry or other patient diagnostic information to determine and/or compare the frequency response. For example, a correction factor based on body geometry may be used. Alternatively or additionally, body geometry may inform system 100 as to which portion of determined signal to use or the like. As discussed herein, body geometry may be entered manually or automatically, and may be determined in part from one or more tools, such as the diagnostic tools 151 discussed in more detail below.

In general, the data analysis unit 161 may receive voltage information related to multiple frequency drive signal, along with the drive signals; drive signals may comprise sequential or simultaneous delivery of 2 or more frequencies. For example, for simultaneously driven signals, the recorded voltages can be split into frequency-correlated components (“bins”) and then analyzed by comparing magnitude/phase of the data in the various frequency “bins”. For example, a 256 pt FFT with 1K bin widths that are centered at the two or more application frequencies may be used. The use of simultaneously driven frequencies may greatly reduce the time to apply/record over all of the electrode/electrode pairs used to calculate the signal and estimate wetness.

Any of the data analysis units 161 described herein may also include a user interface 163. For example, a data analysis unit 161 may include a user output component (e.g. screen) to “report” tissue wetness assessment. Alternatively, the output may be stored, and/or transmitted, e.g. including transmission back to the acquisition module 117 and/or to a separate component such as a third-party database (either with or without concurrent display).

In any of the variations described herein, the output may be an indicator of tissue (e.g., lung) wetness. For example, the apparatus may determine and present a quantitative assessment of lung wetness. The assessment may be a relative indicator, such as a numeric (e.g., 1-10) or qualitative assessment of lung wetness (e.g., dry, somewhat wet, wet, etc.). The assessment may be made for a partial portion of a lung, or an assessment of multiple discrete portions of a lung, or may be generalized to the entire lung, or for one lobe of the lung (or one side of the lung).

As mentioned above, the data analysis unit 161 may also include user interface (e.g., GUI) similar to the user interface described above for the acquisition module 117.

It will be appreciated from the above that the data analysis unit 161 could be of any appropriate form and could include a processing system, such as a suitably programmed PC, Internet terminal, lap-top, or hand-held PC, computer server, or the like. In one example the data analysis unit 161 is a tablet, smart phone, or other portable processing device, that is optionally connected to one or more computer servers, which could be distributed over a number of geographically separate locations, for example as part of a cloud based environment. In this example, the functionality provided by the data analysis unit could be distributed between multiple processing systems and/or devices, depending on the preferred implementation.

In variations including one or more connecting cables, as shown in FIG. 1, the connecting cables may be short. Alternatively, in some variations the apparatus may be configured so that the patch 101 is directly connected to the acquisition module 117, as mentioned above. Alternatively, the connecting cables may be integrated into the patch 101 and/or acquisition module 117.

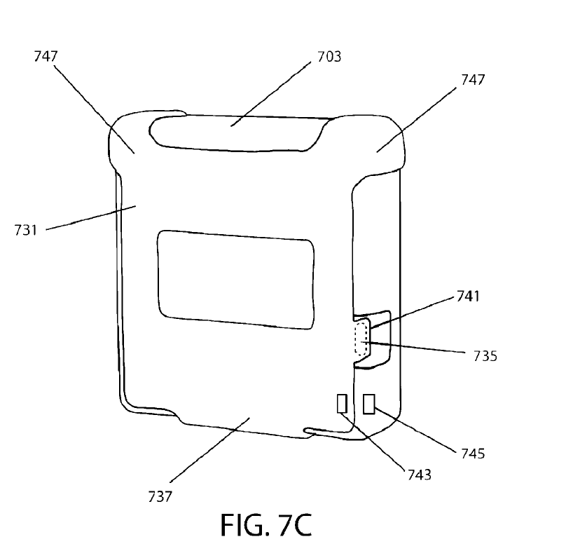

Any of the apparatuses described herein may include one or more wearable holders that may be used to hold some of the components of the apparatus. For example, a patient strap 141 may be used, as shown in FIG. 1. In this variation, the strap may be worn over the subject's shoulder and may include connectors for some of the components. Alternatively or additionally, the wearable holding member (e.g., strap, belt, halter, etc.) may include a Velcro surface to which the components (e.g., acquisition module, battery, etc.) may attach. For example, in some variations, the strap 141 is configured to be positioned over the subject's shoulder when the patient is prone, and the acquisition module 117 may be attached to one side of the strap 141 while the battery (if separate from the acquisition module) may be positioned on the opposite side. In some variations the wearable holding member may be adapted for use with cradle 143, which can be configured similar to cradle 731 shown in FIG. 7C.

In some variations the system does not include a strap. For example, the acquisition module, battery, etc. may be directly (e.g., adhesively) connected to the body, or may be placed near the subject's body, e.g., on a surface such as a bed, table, etc.

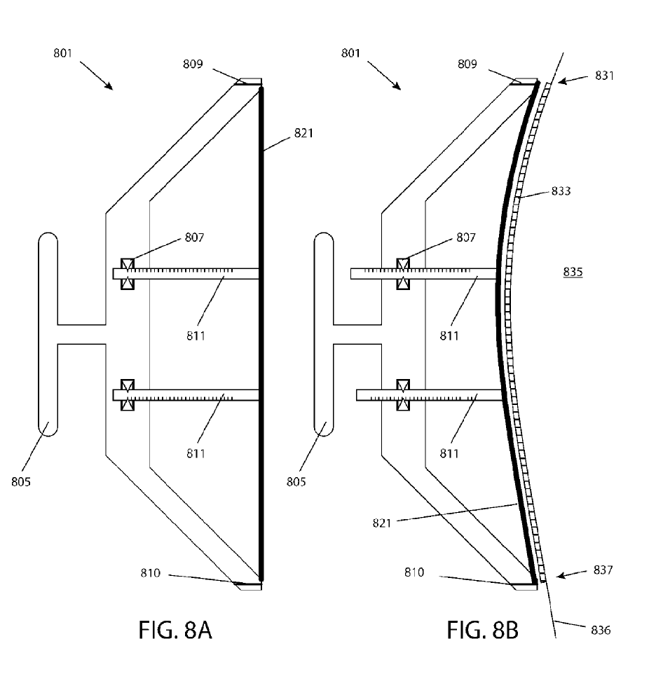

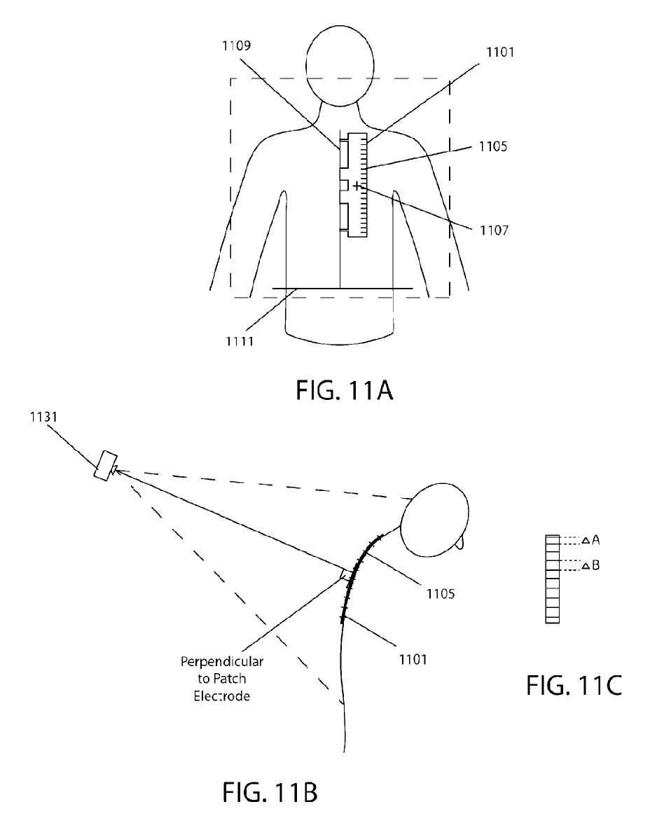

As mentioned above, any of the variations described herein may include a diagnostic tool 151, as will be described in greater detail below. For example, a diagnostic tool may generally be a device to gather patient information. This patient information may be used by the systems (e.g., the data analysis unit 161) to assess tissue wetness. Examples of diagnostic tools include devices to gather back contour information, as illustrated in FIG. 8, 9, 10, 11 or 12 (mechanical or electromechanical measurement devices) and described in greater detail below. Other diagnostic tools may include imaging devices, including devices for performing tissue imaging (e.g., MRI, X-Ray, Ultrasound Imager, etc.). In some variations the imaging device may include a camera. See, for example, FIG. 11, described in greater detail below. For example a camera may be used to take a picture of the subject and/or the setup for calculated estimation of “subject size/curvature”. In some variation the device may include software/firmware/hardware to assist the user in taking the image, so that the user could capture an optimal image. For example, the device may include a heads-up display input (e.g. live guide) to guide the user.

In some variations the apparatus may include control logic that, when executed on a processor causes the device to process the camera image to determine back curvature information. This information may be used to help position the patch and/or correct for patch position when calculating lung wetness. In some variations, the apparatus may include control logic to assist in taking an image (e.g., to guide to user to take an image by providing an orthogonally check, alignment (with patch) check, proper distance from the patient, etc.).

Any of the apparatuses described herein may also include one or more self-diagnostic and/or self-correcting capabilities. For example, U.S. Patent Application Publication No. 2013/0165761 (previously incorporated by reference in its entirety) described a system and method of determining which electrodes 102 to keep/reject when applying stimulation and/or recording signals for determining lung wetness. Such self-diagnostic capability can be incorporated into any of the elements of the apparatus, including the data analysis unit 161 and/or the acquisition module 117 and/or the patch 101.

Diagnostic capabilities may include: applicable patch tests, patch type testing, individual electrode testing (e.g. to determine one or more electrodes 102 “not to be used”, as described above). For example, a voltage may be supplied between an electrode 102 pair (similar to normal operation), and the current measured. If the measured current is within expected range then the electrodes can be determined to be making good contact. If the measured current falls below expected range then it implies the impedance between electrodes is too high, thus poor or no contact. The test may be performed across different combination of pairs of electrodes 102 covering the whole patch. In some instances, a patch 101 with “bad” connections can be used (e.g., if below a maximum) by avoiding using those particular (i.e. identified as bad) electrodes 102 for stimulating and/or sensing.

FIG. 2 illustrates another variation of a patch. In FIG. 2, the patch 101 includes at least a portion of an integrated acquisition module 205. The patch 101 may further include two alignment tabs 107 that may be used to position the array of electrodes relative to patient anatomy. The patch shown in FIG. 2 also includes flexing segments comprising slits 105 to enhance the substrate flexibility when worn on a contoured region of a subject's back, as described above. In addition to the slits in the substrate near the electrodes 102, the sensor patch may also include flexibility enhanced regions 231 (e.g., slits) in the connector tabs 203. Flexibility-enhancing regions (e.g., slits) can be positioned between any or all traces on a connecting tab and/or on the substrate between or otherwise proximate electrodes 102, e.g., between every trace, every 2nd trace, every 3rd trace, etc. If the flexibility enhancing region is a slit, the slit length may be any appropriate length, including the length of the connecting tab, minus clearance space for a connector 209, e.g. in the example shown in FIG. 2, at least 0.25″ (0.64 cm) clearance in an approximately 0.5″ (1.3 cm) long slit. As mentioned above, in this example, the slits are positioned along the lateral edge of the patch on one side (e.g., on the right side in FIG. 2, which would be positioned more laterally offset from the midline of the back on a patient. In FIG. 2, a slit is positioned after every second electrode, though a first slit is positioned between top two electrodes. Alternatively in some variations multiple slits are positioned no more than 2″ (5 cm) apart, e.g., approximately every 0.72″ (1.8 cm). Slits into the lateral side of the patch 101 may extend from (near or at) the lateral edge, and may extend as far as the midpoint (or less) of nearest electrodes. In FIG. 2, the slit has a length of approximately 0.5″ (1.3 cm), such as 0.484″ (1.23 cm). In some variations, the patch 101 includes a slit at each corner of the patch. FIG. 2 shows slits at the superior two corners, however slits could be positioned at any or all of the four corners.

FIG. 3 shows an alternative or additional flexibility-enhancing substrate modification, in which cut-outs 301 through the substrate are included at intervals on the patch to enhance flexibility and allow the patch to better contour a subject's body. The cut-outs (shown as round holes, but could be cut-outs of any appropriate size/shape, including oval, rectangular/square, triangular, diamonds, etc.) may provide stress relief to the substrate of patch 101 during flexing, while allowing the electrodes 102 to remain attached to the subject's body. The cut-out regions may be used as an alternative or in addition to the slits described above.

FIG. 4 illustrates another variation that may be used in addition or as an alternative to the other flexibility-enhancing substrate modifications. In FIG. 4, the patch 101 includes regions, flexing segments that act as flexibility-enhancing substrate modifications to increase flexibility of the patch 101. In this example, the flexing segments 401 may be formed of a more elastic material than neighboring non-flexing portions of the substrate, such as an elastomeric sheet, fabric, weave, or other stretchable material.

Although the variations described above include a substrate for the electrodes 102 that is generally less stretchable than other regions that modify the substrate to enhance flexibility, in some variations the substrate is instead relatively highly flexible, but is treated to provide regions of enhanced stiffness, particularly around the electrodes, to maintain the predetermined relationship (e.g., spacing) between the electrodes. For example, the patch could include a flexible but relatively inelastic spine running substantially along a length of the patch, so that the spacing between electrodes is maintained, whilst allowing the patch to flex and conform to the tissue surface of the subject, for example by allowing the patch to bend along its length or width and/or to allow the patch to twist.

In general, any of the patch 102 variations described herein may also include multiplexing circuitry, for example, to reduce the number of connections to the electrodes.

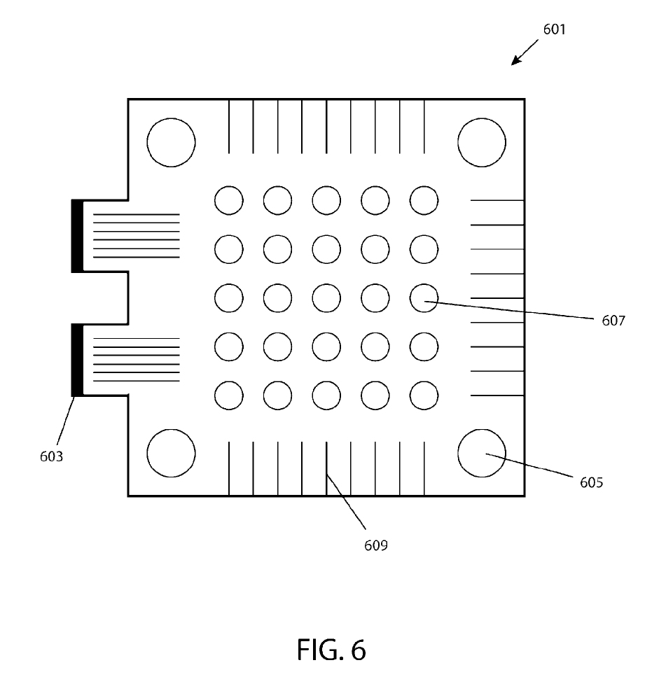

FIG. 5 illustrates another example of a patch, shown in an exploded side view. In this example, the patch 101 has a laminate construction (e.g., is formed of layers connected atop each other). For example, the substrate 503 may be any appropriate material, as described above. In FIG. 5, the traces (connections) 505, 511 may be positioned on the top and bottom surfaces of the substrate 503. The traces may make electrical connection between the electrodes 507 and the other electrical components of the patch 101. The traces may be any appropriate conductive material, such as Ag traces or Ag/AgCl traces. The electrodes 507 may be electrically attached to individual traces on the substrate. In addition, as shown in FIG. 5, the patch may include a dielectric covering 513 (which may be masked at electrode positions), insulating the electrical connectors from the rest of the apparatus and/or the tissue. In some variations, the apparatus may include additional protective coverings 509 over the electrodes, including multiple coverings (e.g., one or more coverings that may be removed in manufacturing or before application to a subject). In some variations the different layers of the patch may be color-coated to enable quick confirmation of the various components of the apparatus. For example, in one variation, the covering may be colored (i.e. not clear) to ease confirmation of its presence.

Another variation of a sensor patch 601 including an array of electrodes is shown in FIG. 6. In FIG. 6, the patch 601 includes a two dimensional (2D) grid of sensing electrodes 607 and four “corner” stimulation (drive) electrodes 605. The sensing electrodes may be used to sense voltage (or current) and the stimulation electrodes may be used to inject current. In FIG. 6, the current injecting electrodes have a larger area than the sensing electrodes. For example, having drive electrodes that are bigger may help introduce current into body at lower voltages than smaller electrodes. In some variations the sensing electrodes may be the same size as the drive electrodes. As in the exemplary patches shown in FIGS. 1 and 2, the patch also includes a connector region 603 for connection to an acquisition module, either directly or indirectly, such as through a connecting cable. In operation, a patch such as the one shown in FIG. 6 may be used to apply current between any two of the stimulation electrodes 605 while voltage may be measured between any of the sensing electrodes 607. In some embodiments, current is injected between one or more of sensing electrodes 607, such as during a diagnostic procedure to detect sufficient contact and/or a calibration procedure to determine a calibration result. In some embodiments, voltage is measured between one or more of electrodes 605, such as during a diagnostic or calibration procedure.

In the exemplary patch shown in FIG. 6, the edges of the patch may also include slits 609 that act as substrate modification (flexibility enhancing) elements. Other variations of substrate modification elements (e.g., holes, regions of more flexible materials, etc.) may be included as well or alternatively.

FIGS. 7A and 7B schematically illustrate how an apparatus such as the apparatus for determining lung wetness shown in FIG. 1 may be worn by a subject. For example, in FIG. 7A, a strap 705 is draped over a subject 739's shoulder and the acquisition module 703 is connected to a patch 701 that has already been attached to the subject's back. The patch may be applied to the subject's back by using the alignment tabs 711 to orient the patch 701 relative to the midline of the back (the patient's spine 733), in order to properly position electrodes 702 for subsequent current delivery and/or voltage measurement. Once the patch 701 is attached (e.g., adhesively attached) to the back of the patient in the correct anatomical location, it may be connected to the acquisition module 703. In FIG. 7B the two are indirectly connected through a pair of connectors. The acquisition module 703 may be held by the strap 705 (or by any other wearable holding member). In FIGS. 7A and 7B, the strap is a wearable holding member to which both the acquisition module 703 and a power supply 707, connected together by a power cord or wire 709 can be connected. In this example, the power supply 707 may counterbalance the weight of the acquisition module 703 when it is worn with the strap over the subject 739's shoulder. As mentioned above, the various components of the apparatus held by the wearable holding member may be secured to the wearable holding member by a button, claps, connector, or the like, including a re-usable connector such as Velcro. For example, the strap may include one side of the Velcro (e.g., the pile side) while the components (such as the power supply, acquisition module, etc.) may include the complimentary (e.g., hook) side of the Velcro.

In some variations one or more components, such as the acquisition module 703, power supply 707, data analysis unit, etc. may be held by an intermediary structure such as a cradle or the like. FIG. 7C illustrates one variation of a cradle 731 that is attachable to (e.g., partially surrounds) an acquisition module 703. In this example, the cradle at least partially surrounds the acquisition module 703 with snap-fit edges on the bottom 737 and two of the corners 747. In this example, the cradle 731 also includes an attachment element (not shown), e.g. Velcro, to attach to the wearable holding member (e.g., strap 705). In addition to holding the component onto the wearable member, a cradle 731 may also provide functional benefits such as protecting various parts of the apparatus from damage or misuse. For example, in FIG. 7C, tab 741 of cradle 731 blocks access to one or more ports 735 (e.g. USB Port) of the acquisition module, which may act as a safety feature. The port may be part of a control access portion that is used in a limited context, such as for uploading/downloading data or otherwise communicate with other portions of the apparatus or other third-party sites, such as a data analysis unit. In some embodiments, one or more ports 735 are only to be used prior or subsequent to positioning of acquisition module 703 proximate the patient (e.g. via strap 705), and tab 741 prevents their use when acquisition module 703 is positioned within cradle 731.

In some variations the apparatus may include additional or alternative attachments to secure components to each other and/or to the wearable holding member. For example, the various components may be magnetically secured to a wearable holding member. A magnetic or other sensor in cradle 731 and/or acquisition module 703 can be configured as a safety interlock, such as to require attachment between the two to allow an apparatus to operate (e.g., drive current). In some embodiments, cradle 731 comprises a sensor 743 and/or acquisition module 703 comprises a sensor 745, each as shown in FIG. 7. Sensors 743 and/or 745 can comprise one or more sensors or transducers, such as one or more mating transducer-sensor pairs, such as a magnet and a magnetic sensor. Sensors 743 and/or 745 can be configured to detect adequate and/or inadequate connection of acquisition module 703 to cradle 731, such as to provide a confirmation of proper attachment that is used to enable further use. Any of the apparatus variations described herein may also include a safety interlock feature, such as described hereabove (e.g., requiring connection between the acquisition module and the cradle and/or the patch sensor and the acquisition module, etc.).

In some variations of a cradle such as the one shown in FIG. 7C, the cradle may include a power supply and/or other electronics.

FIGS. 8A-10 illustrate variations of diagnostic tools as described herein. For example, FIGS. 8A and 8B illustrate one variation of a tool that is configured to measure curvature of a body surface, such as a patient's back or other body surface to which a patch sensor 833 is attached or to be attached, allowing this information to be used when analyzing the impedance measurements.

In one example, the diagnostic tool device includes body extending in an arch from a first contact region to a second contact region, wherein a straight line extending between the first and second contact regions forms a neutral line and a plurality of distance measuring elements coupled to the body and configured to measure the distance from a surface beneath the arch of the body and the neutral line. Thus, this allows the diagnostic tool to be positioned in contact with the subject, and a degree of curvature for the subject's body surface measured. This in turn allows a relative physical geometry of the electrodes to be determined, in turn allowing an accurate electrode separation to be calculated, which can in turn be used when analyzing the measured impedances to determine the wetness.

FIG. 8A shows the diagnostic tool 801, configured as a back curvature measuring device. FIG. 8B shows the tool 801 of FIG. 8A positioned to measure/detect curvature from a patch 833 that is positioned on a patient's back 835. In this example, the diagnostic tool 801 includes a plurality of translating sliders 811 that can be used to estimate back contour data that may be provided (e.g., automatically or manually) to the data analysis unit (not shown) or for separate recording. The tool shown in FIG. 8A includes an indicator/guide 807 to measure the displacement of the sliders 811 and thereby detect how far from flat the flexible member 821 on the back side of the tool is deflected.

For example, in FIG. 8B, the tool is shown pushing against a patch 833 that has been applied to a patient's back 835 against the patient's skin 836, as shown. In FIG. 8B, the tool 801 is centered over patch 833. In general, the tool 801 may be used after or before the patch has been attached to the patient.

In some variations, the tool 801 may include one or more registration mark(s) 809, 810 or other alignment marks/features, e.g. at the ends, that may be used to characteristically position/align the tool 801 relative to the patient and/or the patch 833. For example, an alignment mark on a tool 801 may be aligned with registration marks or features of a patch 833, e.g. aligned with most superior electrode 831 and most inferior electrode 837, and/or other registration mark of a patch 833. In operation, these alignment features may help the tool measure consistent information over time.

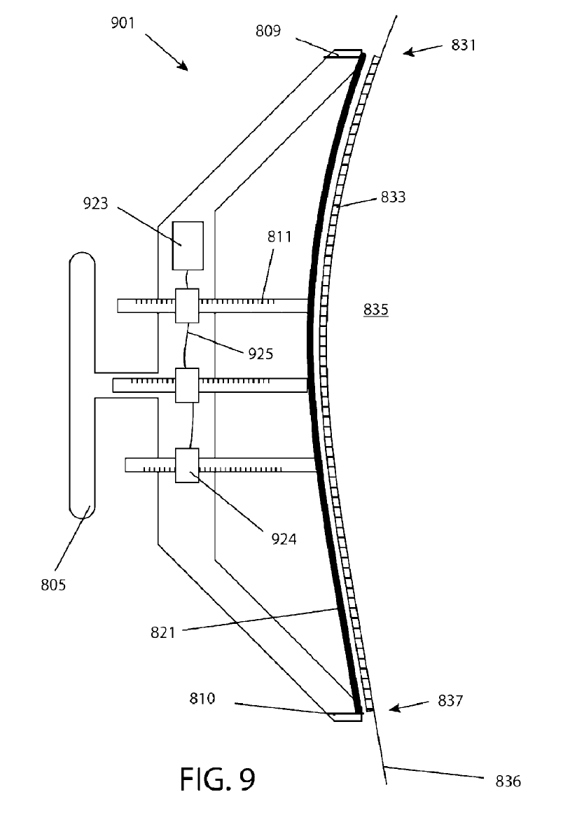

In the curvature measuring tool 801 shown in FIGS. 8A and 8B, the tool generally includes two fixed (rigid) end regions that can be positioned at two points bracketing the surface whose curvature is to be measured. A flexible member 821 (e.g., band, membrane, etc.) may stretch between these fixed points, and the device may measure the deviation of the flexible member 821 from the straight line connection between the two end regions (the ‘neutral’ position). Deviation from the neutral position is measured by the sliders 811. At equilibrium the device maintains the sliders 811 at the neutral position shown in FIG. 8A. When the tool 801 is applied against (e.g., pressed or held against) the surface whose curvature is to be measured, as shown in FIG. 8B, the sliders 811 are deflected from the neutral position (the ends of the sliders 811 may be fixedly attached to the deflectable flexible member 821), so that each slider 811 translates as much as the flexible member 821 is deflected. The final positions of the sliders 811 provide distance measurement information that can be correlated to the curvature of the surface being measured (e.g., the back curvature). As shown in FIG. 9, more than two (e.g., three, four, etc.) sliders 811 can be included as part of the tool for finer resolution.

In the examples shown in FIGS. 8A-9, information may include X points of relative topography data, where X=2 (tool ends)+Y (number of slides). In this example, an estimate of the curvature may be made by approximating the following polynomial:

$$p ( x ) = \frac{{x}^{3} ( {d}_{1} - {d}_{2} )}{84} + \frac{{x}^{2} ( 5 {d}_{2} - 6 {d}_{1} )}{28} + \frac{11 x ( 7 {d}_{1} - 4 {d}_{2} ) {x}^{2}}{84}$$

where

$${d}_{i} = \frac{10 {m}_{i}}{254}$$

for i=1, 2 and x is an element of the set [0, 11]. Using this polynomial, each electrode's elevation can be evaluated; thus, the subject's topography may be approximated based on the two slide measurements taken. The tool may contact just the patch, thereby avoiding touching the patient's back, which may enhance cleanliness/sterility of the procedure, although in some variations the tool may touch the patient's back. The tool may be sterilizable.

In any of the tool variations described herein, the tool may include a display for indicating the measurements or providing feedback to the user (e.g., ready to use, measuring, etc.).

As mentioned above, the data captured by the tool may be manually captured (e.g., visually read and recorded) or it may be automatically or semi-automatically captured. The data may be transmitted to the data analysis unit, where it may be used in determining lung wetness, and/or stored for future reference, and/or transmitted. In some variations, the information recorded by the tool may be sent via a wired or wireless communication to the data analysis unit. In some variations, the apparatus may include a control or input, such as a button, knob, trigger, etc., to initiate capture of the measurement and/or storage or transmission of the values measured. For example a button or other control may be located on the handle 805.

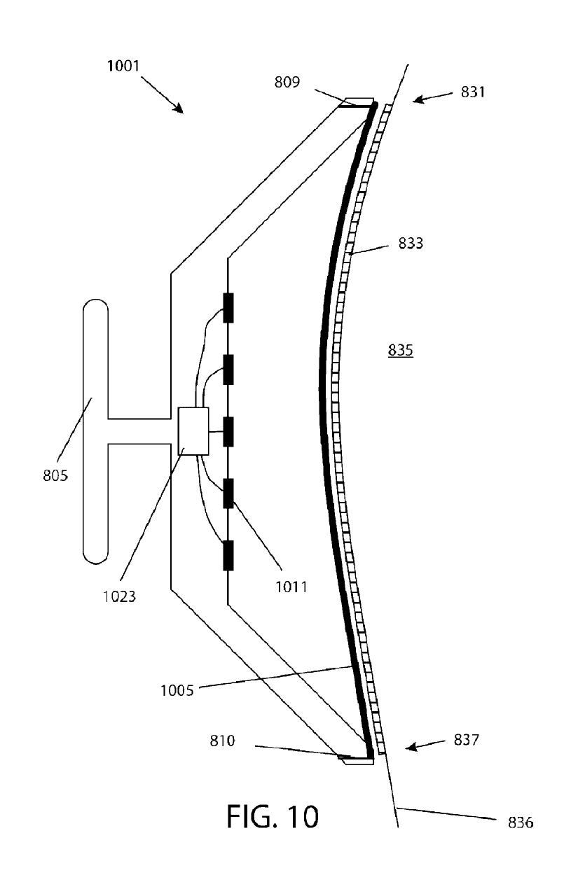

In some variations, the tool may include translating slides that pass through an electronic measurement element, as illustrated in FIG. 9. In FIG. 9, tool 901 includes a plurality of electronic readers 924 that may measure the position of the sliders 811; an electronics module 923 may be used to capture this material for storage/reading/transmission, as mentioned above. Readers 924 can be interconnected, as well as connected to electronics module 923 via one or more cables 925. In some variations, other non-contacting (e.g., distance) measurement elements may be used to determine curvature. For example, FIG. 10 illustrates one variation of a tool 1001 in which a plurality of optical distance sensors 1011 measure the displacement of the flexible member 1005 due to the curvature of the underlying surface (the patch 833 on the skin surface 836 of the subject's back 835). The optical sensors may communicate with an electronics module 1023 for display, transmission or storage of the measured values. One or more controls (not shown) may be included, e.g. on the handle 805. In FIG. 10, the optical sensors 1011 may be used with or without a flexible member 1005, as they may directly measure the distance to the outer surface of the patch and/or the patient's back. This variation may otherwise be used as described above for the tools of FIGS. 8A-8B and 9. For example an upper alignment mark 809 may be positioned on or adjacent to the most superior electrode 831 of the patch (or some other landmark on the patch or patient's body), and the lower alignment mark 810 may be positioned on or adjacent to a second landmark on the patch or patient's body (e.g., the most inferior electrode 837, as show in FIG. 10), and the measurements made and transmitted to the data analysis unit.

FIGS. 11A and 11B illustrate operation of another non-contact measurement tool, which may directly image a patch (or other markings) on the subject's back to determine curvature information. In FIG. 11A, a subject's back is shown, with a patch sensor (patch 1101) positioned offset from the subject's spine 1109, such as by using alignment tabs as mentioned above. The patch 1101 includes markings identifying the center of the patch 1101, center markings 1107, as well as fiducial markings 1105 along the lateral edge of the patch. In general, any number of markings along one or more portions of the patch 1101 may be used, which may be identified by the imaging system (e.g., camera 1131 in FIG. 11B). As shown in FIG. 11B, a diagnostic tool comprising camera 1131 is positioned at approximately 90° relative to the surface of the patch 1101, and can image the markings 1105 on the patch 1101 and may measure the relative distance between the markings 1105 to determine curvature of the back, as illustrated in FIG. 11C.

This variation of a non-contacting image analyzer may therefore work with markings on the patch to determine curvature of back. As mentioned, in FIGS. 11A and 11B, the diagnostic tool comprises a camera-based device used to measure curvature of the subject's back. The patch 1101 includes markings which are visualized/identified by the camera-device 1131. In some variations, the tool images the patient's back directly and determines (e.g., by focus or by optical sounding) the changes in back curvature. As shown in FIGS. 11B and 11C, when the markings are spaced equidistantly or at any known separation distance, the camera image may correlate distance between markings to curvature of back. The resolution of the curvature detection may be increased by increasing the number of markings detected. For example, markings may be made after each electrode, every 2nd, every 3rd, etc. Foreshortening of the distance between the markings on the image may correlate to the curvature of the surface (e.g., patient's back) being examined. As mentioned above, in FIG. 11C, the distance between the first set of markings (e.g., ΔA) appears shorter than between the next set of markings (ΔB) due to curvature at that location. Any of the non-contact tools described herein may include a “calibration” step, e.g., using a calibration image, to compare to, for example, by placing the tool against a flat surface or otherwise known contour surface, taking measurements from a known surface and/or imaging a known surface. Also, as mentioned above, in FIG. 11B, the camera 1131 can be positioned so that it can detect the relative change in the curvature from a known relationship to the patch. For example, the camera 1131 may be positioned orthogonal to the center of an electrode of the patch 1101 (as marked), the electrode of the patch 1101 may be centered in the field of view of the camera/image. The overall field of view may include the bottom of the subject's head to the subject's waist 1111, and the full torso width.

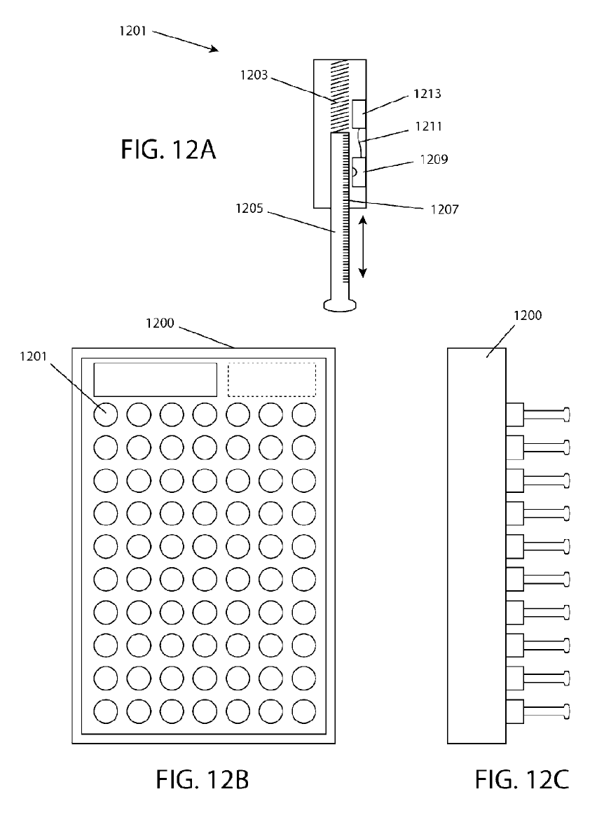

Another variation of a tool for measuring the surface (e.g. curvature) onto which the patch is connected may be illustrated in FIGS. 12A-12C. In this variation, as shown in FIGS. 12B-12C, the tool 1200 may include a non-linear array of translating slides or other distance measurement elements. The distance measurement elements (e.g., sliders) may be arranged in a grid (e.g., a 2D grid) as shown in FIG. 12B. For example in FIG. 12A, a distance transducer assembly 1201 includes a plunger 1205 connected to a bias (spring 1203); the plunger includes markings 1207 that can be read automatically (in this example) using a visual sensor 1209 that is connected through a connector 1211 to an electronics module 1213. By arranging an array of these transducer assemblies 1201, as shown in FIGS. 12B and 12C (in which all of the assemblies 1201 are connected to an electronics module 1213), the tool 1200 may be used to detect the surface of a two-dimensional region. In this example, the diagnostic tool 1200 (“back curvature device”) includes the 2D array of plunger-based transducer assemblies 1201 discussed above, and may be used to provide multi-point 2D topography of a subject's body, including the back region.

As illustrated in FIG. 1, any of the tools described herein (including those in FIGS. 8A-12C) may be included as part of the apparatus (e.g., system 100) for determining tissue wetness, including lung wetness. In some variations, the tool may be integrated into or onto the patch. For example, the patch may determine the shape of the region of the patient's body onto which it is placed, and a separate tool is not necessary. In some embodiments, another component of system 100 is configured to determine the shape of the region of the patient's body onto which the patch is placed, such as the acquisition module 117, strap 141 and/or cradle 143.

FIG. 13 illustrates one method of determining tissue wetness using an apparatus as described herein. In particular, FIG. 13 illustrates a method of attaching a patch and performing a tissue wetness assessment. Before attaching the patch, the skin may be prepared in step 1310, for example, by exposing, washing, cleaning, etc., so that the skin is ready to receive the patch. At least the area to be covered by the patch (the placement area) may be prepped. The skin may be exfoliated, for example, using a skin prep tape, while avoiding scratching the skin. The skin may also be cleaned (e.g., using an alcohol wipe). In variations in which lung wetness is to be examined, the skin may be prepared about 1″ (2.5 cm) below the top of the shoulder and immediately lateral (e.g., to the right of) the spine down to about 13″ (33 cm) down the back (e.g., 1″ (2.5 cm) beyond the lowest patch or electrode location), and about 5″ (12.7 cm) lateral (e.g., to the right of or to the left of) the spine (e.g., about 1″ (2.5 cm) beyond right-most patch or electrode location). Although in general the patch may be placed to the right of the spine (e.g., the patient's right lung) in some variations the method may instead place the sensor on the left side (e.g., over the patient's left lung). In some variations, preparation may include removing hair from at least the patch placement area.

Once the area is prepared, the wearable holding element (e.g., strap) may be placed on (worn by) the subject in step 1320. The acquisition module may be attached to the holding element (e.g., within a cradle) or it may be attached later. For example, a strap may be laid over the subjects shoulder (e.g., the left shoulder). As mentioned above, a power supply may be positioned on the front side of the strap, and the acquisition module may be attached to the back. In variations in which the battery is integrated with the acquisition module, nothing (or a dummy weight) may be attached to the front.

The more precise location for placement of the patch may then be determined. For example, when measuring lung wetness, the spine location that may be used to guide positioning of the patch over the lung may be determined in step 1330. For example, the back of the patient may be palpated to find the specific vertebral location (e.g., T2), which can be identified, for example, by having the patient lower their head (chin to chest). The subject can then be asked to look up/down, left/right—to confirm proper identification at T2 (no motion should be present). A mark may be made (e.g., with a non-toxic, washable marker) and used to orient the patch. The patch may then be positioned in step 1340, such as by positioning a superior end and/or a superior alignment tab of the patch relative to T2. Thereafter, the patch may be applied in step 1360. For example, the patch may be positioned with the superior alignment tab at the upper spine location and the other tabs oriented with the other alignment tabs. At least a portion (or all) of a protective covering of electrodes (backing) can be removed, and the patch applied directly to the skin while maintaining the location of the superior alignment tab. In some variations the inferior alignment tab may aligned with the spine in the proper location in step 1350 before adhering the patch to the skin in step 1360. For example, the top-left portion of patch may be adhered at location identified in step 1330, so that a superior-inferior location as well as a medial-lateral location of the patch are properly located on the patient's back.

When adhering the patch in step 1360, the patch may be slowly laid down, electrode by electrode, being careful to maintain the alignment (e.g., vertical alignment), while avoiding tugging of the patch in a way that may cause one or more electrodes (gel) to shift. Gentle pressure may be applied to each electrode, though large forces that may flatten the electrode (gel) should be avoided. Buckling of the patch should also be avoided, so that the orientation of the electrodes on the patch relative to each other may be maintained. In some variations, the patch may be removed and repositioned to remove or otherwise avoid buckling, misalignment, etc. For patients with kyphosis (e.g., curvature toward slouching or hunchback), the patch may be placed farther laterally (e.g., farther to the right) to compensate for the tendency for diagonal placement in such patients. For patients with scoliosis (e.g., sideways curvature of the spine), the patch may be positioned to best align with the underlying lung while avoiding placement over the spine and scapula. For patients with excessive fat deposits or skin folds, the patch may be placed by following the curve of the skin fold and apply extra pressure during adhesion (making sure to prepare all of the skin, including the skin in skin fold(s)). In placing the patch on patients with skin folds, the user may avoid placing the patch in regions with folds that are too large or to deep.

Once the patch has been placed, in some variations a diagnostic assessment of the patch placement may be performed in step 1365. For example, as described above, information about the patient may be collected, including but not limited to the body contour information. For example, a tool as described above may be used to perform patient diagnostics to collect information. This step may be repeated to collect additional data, or to refine the data collected. As mentioned above, in any of the variations described herein, the patient's body contour information may be collected before applying the patch (e.g., up to a day or more before applying the patch) or after collecting electrical properties data in step 1380.

Once the patch is attached to the patient, it may be connected to the acquisition module in step 1370. In some variations, the patch may be collected to the acquisition module before the patch is attached to the patient's skin. More often, the patch is attached to the acquisition module after it has been attached to the skin. In the variation shown in FIG. 1, two connections are made between the acquisition module and the patch. Alternatively, the patch may be pre-attached to the acquisition module. In some variations, after the patch is connected a self-diagnostic run may be made to confirm the proper attachment (e.g. proper electrical attachment), and/or to determine that the patch is “good”.

Once the patch is attached, data may be collected by driving power through the electrodes and sensing the resulting response of the tissue in step 1380. For example, voltage data between two or more pairs of electrodes may be collected while driving current from other pairs of electrodes (e.g., at one or more frequencies as described above). Thus, multiple currents may be driven at different frequencies simultaneously or sequentially. Data may be collected as described in U.S. 2013/0165761 from multiple pairs of sensing electrodes. This data may be gathered, processed (e.g., filtered, averaged, etc.) and transmitted, stored and/or analyzed, for example, by a data analysis unit in step 1390 to determine tissue wetness.

For example, the process may be performed to determine a lung wetness assessment that is qualitative and/or quantitative and may provide output either directly (e.g., on a display on the apparatus) and/or indirectly, e.g., by transmitting to a physician, patient medical record, or the like. In some variations, the wetness assessment may be performed without using body geometry information. Alternatively, in some variations, the wetness assessment may be performed using body geometry information.

In one example, wetness assessment is performed to determine a wetness indicator, which could be a numerical value or graphical representation of an absolute wetness, or wetness relative to a baseline or other reference. For example, wetness measurements could be obtained for individuals in a reference population, with comparison to the wetness measurements being used to determine the wetness indicator, indicating whether the wetness is greater or less than desirable/expected which could in turn be indicative of a medical condition associated with the wetness, or the like.

Thus, in one example, a method of determining tissue wetness includes attaching a patch sensor comprising a plurality of drive electrodes and sensing electrodes to a skin surface of a subject's body, measuring a curvature of the skin surface of the subject's body, applying drive currents at a plurality of different frequencies to the drive electrodes and measuring voltages at a plurality of different sensing electrodes, determining an estimate of electrical properties for a plurality of regions beneath the patch sensor using the applied drive currents and measured voltages and determining an estimate of tissue wetness from a frequency response of the determined electrical properties.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

1. A non-invasive patch sensor, the patch sensor comprising:
a substrate;
a plurality of electrodes on the substrate, wherein the substrate maintains a predetermined spacing between the electrodes; and
at least one substrate modification to enhance local flexibility of the substrate so that the patch sensor may conform to a contour of a subject's body,
wherein the plurality of electrodes are configured to form a plurality of pairs of current-injecting electrodes and a plurality of pairs of voltage detection electrodes.

2. The patch sensor of claim 1, wherein the substrate modifications to enhance local flexibility of the substrate comprise at least one of:
cut-out regions through the substrate;
slits cut through the substrate; and,
regions of material within the substrate having a greater flexibility than the substrate.

3. The patch sensor of claim 1, wherein the substrate at least one of:
a. is flexible and relatively inelastic, so that the spacing between each of the electrodes remains relatively fixed as the sensor is manipulated;
b. is less than about 5 mils (0.127 mm) thick;
c. comprises at least one of:
a polyester material; and
a polyester material and an anti-bacterial titanium oxide material;
d. has a width of between about 0.5 inches (1.3 cm) and about 2.5 inches (6.4 cm).

4. The patch sensor of claim 1, further comprising an adhesive hydrogel.

5. (canceled)

6. (canceled)

7. (canceled)

8. The patch sensor of claim 1, wherein the plurality of electrodes comprise more than 6 elongate electrodes each having a length of between about 1.5 inches (3.8 cm) and about 2.5 inches (6.4 cm) and a width of between about 0.1 inches (0.3 cm) and about 0.5 inches (1.3 cm), wherein the electrodes are arranged with their lengths perpendicular to a proximal to distal axis on a subject-contacting surface of the substrate so that the electrodes extend in a line parallel to the proximal to distal axis of the substrate to form an active region that extends between about 6 inches (15 cm) and about 14 inches (36 cm) along the proximal to distal axis.

9. The patch sensor of claim 1, wherein the plurality of electrodes comprise at least one of:
more than 10 electrodes; and,
more than 25 electrodes.

10. The patch sensor of claim 1, wherein the electrodes at least one of:
a. have a rectangular shape on the substrate;
b. comprise silver/silver chloride electrodes; and,
c. are separated by a fixed distance of between about 0.2 and about 0.5 inches on center down a proximal to distal length of the substrate.

11. (canceled)

12. (canceled)

13. A non-invasive patch sensor, the patch sensor comprising:
a substrate;
a plurality of electrodes on the substrate, wherein the substrate maintains a predetermined spacing between the electrodes; and
a plurality of alignment tabs extending from a lateral side of the substrate wherein the alignment tabs are between about 0.2 inches (0.5 cm) and about 2 inches (5 cm) long and greater than about 0.1 inches (0.3 cm) wide,
wherein the plurality of electrodes are configured to form a plurality of pairs of current-injecting electrodes and a plurality of pairs of voltage detection electrodes.

14. The patch sensor of claim 13, wherein the alignment tabs at least one of:
a. are between about 0.1 inches (0.3 cm) and about 3 inches (7.6 cm) wide;
b. comprise an upper alignment tab and a lower alignment tab.

15. (canceled)

16. The patch sensor of claim 13, wherein the substrate at least one of:
a. is flexible and relatively inelastic, so that the spacing between each of the electrodes remains relatively fixed as the sensor is manipulated;
b. is less than about 5 mils (0.127 mm) thick;
c. comprises a polyester material; and,
d. has a width of between about 0.5 inches (1.3 cm) and about 2.5 inches (6.4 cm), not including the width of the alignment tabs.

17. The patch sensor of claim 13, further comprising an adhesive hydrogel.

18. (canceled)

19. (canceled)

20. (canceled)

21. The patch sensor of claim 13, wherein the plurality of electrodes comprise more than 6 elongate electrodes each having a length of between about 1.5 inches (3.8 cm) and about 2.5 inches (6.4 cm) and a width of between about 0.1 inches (0.3 cm) and about 0.5 inches (1.3 cm), wherein the electrodes are arranged with their lengths perpendicular to a proximal to distal axis on a subject-contacting surface of the substrate so that the electrodes extend in a line parallel to the proximal to distal axis of the substrate to form an active region that extends between about 6 inches (15 cm) and about 14 inches (36 cm) along the proximal to distal axis.

22. The patch sensor of claim 13, wherein the plurality of electrodes comprise at least one of:
more than 10 electrodes; and,
more than 25 electrodes.

23. The patch sensor of claim 13, wherein the electrodes at least one of:
a. have a rectangular shape on the substrate;
b. comprise silver/silver chloride electrodes; and,
c. are separated by a fixed distance of between about 0.2 inches (0.5 cm) and about 0.5 inches (1.3 cm) on center down a proximal to distal length of the substrate.

24. (canceled)

25. (canceled)

26. A diagnostic tool device for measuring the surface contour of a region of a patient's body, the diagnostic tool comprising:
a body extending in an arch from a first contact region to a second contact region, wherein a straight line extending between the first and second contact regions forms a neutral line; and
a plurality of distance measuring elements coupled to the body and configured to measure the distance from a surface beneath the arch of the body and the neutral line.

27. The device of claim 26, further comprising at least one of:
a. a flexible member extending between the first contact region and second contact region;
b. a handle opposite the arch; and,
c. a first alignment mark on the first contact region and a second alignment mark on the second contact region.

28. (canceled)

29. (canceled)

30. The device of claim 26, wherein the distance measuring elements comprise at least one of:
a. sliders configured to be pushed by the surface beneath the arch of the body
b. sliders coupled to a flexible member extending between the first contact region and second contact region; and,
c. non-contact, optical distance measuring elements.

31. (canceled)

32. The device of claim 26, further comprising a plurality of guides on the body configured to provide an estimate of distance based on the deflection of the distance measuring elements.

33. The device of claim 26, further comprising an electronic reader configured to read measurements from the distance measuring elements.

34. (canceled)

35. A method of determining tissue wetness, the method comprising:
attaching a patch sensor comprising a plurality of drive electrodes and sensing electrodes to a skin surface of a subject's body;
measuring a curvature of the skin surface of the subject's body;
applying drive currents at a plurality of different frequencies to the drive electrodes and measuring voltages at a plurality of different sensing electrodes;
determining an estimate of electrical properties for a plurality of regions beneath the patch sensor using the applied drive currents and measured voltages; and
determining an estimate of tissue wetness from a frequency response of the determined electrical properties.
